(19) United States
(12) Patent Application Publication
(10) Pub. No.: US 2007/0245810 A1
(43) Pub. Date: Oct. 25, 2007

(54) Detection Cartridges, Modules, Systems and Methods

(76) Inventors: Chad J. Carter, Lake Elmo, MN (US); Larry H. Dodge, River Falls, WI (US); Samuel J. Gason, Caulfield North (AU); Raymond P. Johnston, Lake Elmo, MN (US); Jeffrey D. Smith, Marine on St. Croix, MN (US); Kenneth B. Wood, Minneapolis, MN (US)

Correspondence Address: 3M INNOVATIVE PROPERTIES COMPANY, PO BOX 33427, ST. PAUL, MN, 55133-3427, US

(21) Appl. No.: 10/596,948

(22) PCT Filed: Dec. 17, 2004
(86) PCT No.: PCT/US04/42455

§ 371 Date: Apr. 2, 2007

Related U.S. Application Data
(60) Provisional application No. 60/533,169, filed on Dec. 30, 2003.

(51) Int. Cl. G01N 33/487

(52) U.S. Cl. 73/53.01

(57) ABSTRACT

Detection cartridges and associated components, as well as methods of using the same that provide sample materials to a sensor for detection are disclosed. Among the components that may be used in connection with the detection cartridges of the present invention are, e.g., input modules, fluid flow front control features, and volumetric flow rate control features. The modules may include one or more chambers containing different constituents for mixing and/or delivery into a detection cartridge.

### RELATED APPLICATIONS

The present application claims priority to U.S. Provisional Patent Application Ser. No. 60/533,169, filed on Dec. 30, 2003, which is hereby incorporated by reference in its entirety.

### GOVERNMENT RIGHTS

The U.S. Government may have certain rights to this invention under the terms of DAAD 13-03-C-0047 granted by Department of Defense.

The present invention relates detection cartridges and methods for detecting one or more target analytes in fluid sample material.

Unlike classical clinical assays such as tube and slide coagulase tests, the detection cartridges of the present invention employ an integrated sensor. As used herein “sensor” refers to a device that detects a change in at least one physical property and produces a signal in response to the detectable change. The manner in which the sensor detects a change may include, e.g., electrochemical changes, optical changes, electro-optical changes, acousto-mechanical changes, etc. For example, electrochemical sensors utilize potentiometric and amperometric measurements, whereas optical sensors may utilize absorbance, fluorescence, luminescence and evanescent waves.

One technical problem that may be associated with many sensors is that the flow rate and/or flow front progression across the detection surface of a sensor may affect accurate detection of target analytes. Control over both volumetric flow rate and fluid flow front progression may, however, be difficult if the detection surface of the sensor is flat because such surfaces may be subject to the formation of voids, bubbles, etc. due to surface tension in liquids moving across a such a surface. Although some sensors may be adapted to address these concerns by including detection surfaces that are not flat and/or featureless, others, such as, e.g., acousto-mechanical sensors, may preferably include a relatively flat, featureless detection surface to function well.

Many biological analytes are introduced to the sensors in combination with a liquid carrier. The liquid carrier may undesirably reduce the sensitivity of the acousto-mechanical detection systems. Furthermore, the selectivity of such sensors may rely on properties that cannot be quickly detected, e.g., the test sample may need to be incubated or otherwise developed over time. Selectivity can, however, be obtained by binding a target biological analyte to, e.g., a detector surface.

Selective binding of known target biological analytes to detector surfaces can, however, raise issues when the sensor used relies on acousto-mechanical energy to detect the target biological analyte due to the size and relative low level of mechanical rigidity of many or most biological analytes. This issue may be especially problematic in connection with shear-horizontal surface acoustic wave detection systems.

Shear horizontal surface acoustic wave sensors are designed to propagate a wave of acousto-mechanical energy along the plane of the sensor detection surface. In some systems, a waveguide may be provided at the detection surface to localize the acousto-mechanical wave at the surface and increase the sensitivity of the sensor (as compared to a non-wave-guided sensor). This modified shear horizontal surface acoustic wave is often referred to as a Love-wave shear horizontal surface acoustic wave biosensor (“LSH-SAW”).

Such sensors have been used in connection with the detection of chemicals and other materials where the size of the target analytes is relatively small. As a result, the mass of the target analytes is largely located within the effective wave field of the sensors (e.g., about 60 nanometers (nm) for a sensor operating at a frequency of 103 Megahertz (MHz) in water).

What has not heretofore been appreciated is that the effective wave field of the sensors is significantly limited relative to the size of biological analytes to be detected. For example, biological analytes that are found, e.g., in the form of single cell microorganisms, may have a typical diameter of, e.g., about 1 micrometer (1000 nm). As noted above, however, the effective wave field of the sensors is only about 60 nm. As a result, significant portions of the mass of the target analyte may be located outside of the effective wave field of the sensors.

In addition to the size differential, the target biological analytes are often membranes filled with various components including water. As a result, the effect of acousto-mechanical energy traveling within the effective wave field above a sensor on the total mass of the biological analytes can be significantly limited. In many instances, target biological analytes attached to the surfaces of such sensors cannot be accurately distinguished from the liquid medium used to deliver the agents to the detector.

Although not wishing to be bound by theory, it is theorized that the relative lack of mechanical rigidity in biological analytes attached to a detection surface, i.e., their fluid nature, may significantly limit the amount of mass of the biological analytes that is effectively coupled to the detection surface. In other words, although the biological analytes may be attached to the detection surface, a significant portion of the mass of the biological analyte is not acoustically or mechanically coupled to the acousto-mechanical wave produced by the sensor. As a result, the ability of an acousto-mechanical biosensor (e.g., a LSH-SAW biosensor) to effectively detect the presence or absence of target biological analytes can be severely limited.

### SUMMARY OF THE INVENTION

The present invention provides detection cartridges and associated components, as well as methods of using the same that provide sample materials to a sensor for detection. Among the components that may be used in connection with the detection cartridges of the present invention are, e.g., input (or fluid) modules, fluid flow front control features, volumetric flow rate control features, etc.

Potential advantages of the apparatus and methods of the present invention are the presentation of sample materials (which may include, e.g., test specimens, reagents, carrier fluids, buffers, etc.) to the detection surface of a sensor in a controlled manner that may enhance detection and/or reproducibility.

The controlled presentation may include control over the delivery of sample material to the detection surface. The control may preferably be provided using a module-based input system in which sample materials such as, e.g., test specimens, reagents, buffers, wash materials, etc. can be introduced into the detection cartridge at selected times, at selected rates, in selected orders, etc.

Controlled presentation may also include control over the fluid flow front progression across the detection surface. The “flow front”, as that term is used herein, refers to the leading edge of a bolus of fluid moving across a detection surface within a detection chamber. A potential advantage of control over the flow front progression is that preferably all of the detection surface may be wetted out by the sample material, i.e., bubbles or voids in the fluid that could occupy a portion of the detection surface may preferably be reduced or eliminated.

Controlled presentation may also encompass volumetric flow control through a detection chamber that, in some embodiments of the present invention, may be achieved by drawing fluid through the detection chamber using, e.g., capillary forces, porous membranes, absorbent media, etc. Controlling the flow rate of sample material over the detection surface may provide advantages. If, for example, the flow rate is too fast, target analyte in the sample material may not be accurately detected because selective attachment may be reduced or prevented. Conversely, if the flow rate is too slow, excessive non-specific binding of non-targeted analytes or other materials to the detection surface may occur, thereby potentially providing a false positive signal. The present invention also provides sealed modules that may be selectively incorporated into, e.g., a detection cartridge, to facilitate the detection of different target analytes within the detection cartridge. Before use, the modules may preferably be sealed to prevent materials located therein from escaping and/or to prevent contamination of the interior volume of the modules. The modules may preferably include two or more isolated chambers in which different constituents may be stored before they are introduced to each other and to the detection cartridges. The introduction and mixing of the different constituents, along with their introduction into the detection cartridge (and, ultimately, the sensor) may be controlled using the modules and actuators. Isolated storage of many different reagents may greatly enhance the shelf-life of materials that may be used to assist in the detection of target analytes. Some reagents that may benefit from isolated dry storage conditions may include, e.g., lysing reagents, fibrinogen, assay-tagged magnetic particles, etc.

The modules may be selected and attached to the detection cartridge by the manufacturer or, in some instances, by an end user. The flexibility offered to an end user to, essentially, customize a detection cartridge at the point-of-use may offer additional advantages in terms of economy and efficiency. For example, different modules containing different reagents, buffers, etc. may be supplied to the end-user for their selective combination of modules in a detection cartridge to perform a specific assay for a specific target analyte.

The detection cartridges of the present invention may incorporate a wide variety of sensors to detect one or more target analytes. The sensors may preferably be in the form of biosensors, where “biosensors” are sensors adapted to detect one or more target biological analytes in sample material.

Although the exemplary embodiments described herein may include a single sensor, the detection cartridges of the present invention may include two or more sensors, with the two or more sensors being substantially similar to each other or different. Furthermore, each sensor in a detection cartridge according to the present invention may include two or more channels (e.g., a detection channel and a reference channel). Alternatively, different sensors may be used to provide a detection channel and a reference channel within a detection cartridge. If multiple sensors are provided, they may be located in the same detection chamber or in different detection chambers within a detection cartridge.

The sensors used in connection with the detection cartridges of the present invention may rely on a wide variety of different sensor technologies. Examples of some potentially useful sensor technologies may include, but are not limited to, sensing electrochemical changes, optical changes, electro-optical changes, acousto-mechanical changes, etc.

It may be preferred that the detection cartridges detect the presence of target analytes in the sample material using acousto-mechanical energy generated by a sensor-located within the cartridge. The acousto-mechanical energy may preferably be provided using an acousto-mechanical sensor, e.g., a surface acoustic wave sensor such as, e.g., a shear horizontal surface acoustic wave sensor (e.g., a LSH-SAW biosensor), although other acousto-mechanical sensor technologies may be used in connection with the systems and methods of the present invention in some instances.

It may be preferred that the detection cartridges and modules of the present invention be designed to detect target analytes that are biological in nature, e.g., target biological analytes. As used herein, “target biological analyte” may include, e.g., microorganisms (e.g., bacteria, viruses, endospores, fungi, protozoans, etc.), proteins, peptides, amino acids, fatty acids, nucleic acids, carbohydrates, hormones, steroids, lipids, vitamins, etc.

The target biological analyte may be obtained from a test specimen that is obtained by any suitable method and may largely be dependent on the type of target biological agent to be detected. For example, the test specimen may be obtained from a subject (human, animal, etc.) or other source by e.g., collecting a biological tissue and/or fluid sample (e.g., blood, urine, feces, saliva, semen, bile, ocular lens fluid, synovial fluid, cerebral spinal fluid, pus, sweat, exudate, mucous, lactation milk, skin, hair, nails, etc.). In other instances, the test specimen may be obtained as an environmental sample, product sample, food sample, etc. The test specimen as obtained may be a liquid, gas, solid or combination thereof.

Before delivery to the detection cartridge and/or modules of the present invention, the test specimen may be subjected to prior treatment, e.g., dilution of viscous fluids, concentration, filtration, distillation, dialysis, addition of reagents, chemical treatment, etc.

The present invention may be utilized in combination with various materials, methods, systems, apparatus, etc. as described in various U.S. and PCT patent applications identified below, all of which are incorporated by reference in their respective entireties. They include U.S. patent application Ser. No. 60/533,162, filed on Dec. 30, 2003; U.S. patent application Ser. No. 60/533,178, filed on Dec. 30, 2003; U.S. patent application Ser. No. 10/896,392, filed Jul. 22, 2004; U.S. patent application Ser. No. 10/713,174, filed Nov. 14, 2003; U.S. patent application Ser. No. 10/987,522, filed Nov. 12, 2004; U.S. patent application Ser. No. 10/714,053, filed Nov. 14, 2003; U.S. patent application Ser. No. 10/987,075, filed Nov. 12, 2004; U.S. patent application Ser. No. 60/533,171, filed Dec. 30, 2003; U.S. patent application Ser. No. 10/960,491, filed Oct. 7, 2004; U.S. patent application Ser. No. 60/533,177, filed Dec. 30, 2003; U.S. patent application Ser. No. 60/533,176, filed Dec. 30, 2003; U.S. patent application Ser. No. 60/533,169, filed Dec. 30, 2003; U.S. patent application Ser. No. ______, titled “Method of Enhancing Signal Detection of Cell-Wall Components of Cells”, filed on even date herewith (Attorney Docket No. 59467US002); U.S. patent application Ser. No. ______, titled “Soluble Polymers as Amine Capture Agents and Methods”, filed on even date herewith (Attorney Docket No. 59995US002); U.S. patent application Ser. No. ______, titled “Multifunctional Amine Capture Agents”, filed on even date herewith (Attorney Docket No. 59996US002); PCT Application No. ______, titled “Estimating Propagation Velocity Through A Surface Acoustic Wave Sensor”, filed on even date herewith (Attorney Docket No. 58927WO003); PCT Application No. ______, titled “Surface Acoustic Wave Sensor Assemblies”, filed on even date herewith (Attorney Docket No. 58928WO003); PCT Application No. ______, titled “Acousto-Mechanical Detection Systems and Methods of Use”, filed on even date herewith (Attorney Docket No. 59468WO003); and PCT Application No. ______, titled “Acoustic Sensors and Methods”, filed on even date herewith (Attorney Docket No. 60209WO003).

In one aspect, the present invention provides a detection cartridge that includes a housing with an interior volume; a sensor operably attached to the housing, the sensor including a detection surface; a detection chamber located within the interior volume of the housing, wherein the detection chamber has a volume defined by the detection surface and an opposing surface spaced apart from and facing the detection surface, wherein the opposing surface includes a flow front control feature; and a waste chamber located within the interior volume of the housing, the waste chamber in fluid communication with the detection chamber.

In another aspect, the present invention provides a detection cartridge that includes a housing with an interior volume; a sensor operably attached to the housing, the sensor including surface acoustic wave acousto-mechanical sensor; a detection chamber located within the interior volume of the housing, wherein the detection chamber has a volume defined by the detection surface and an opposing surface spaced apart from and facing the detection surface, wherein the opposing surface includes one or more channels formed therein; a waste chamber located within the interior volume of the housing, the waste chamber in fluid communication with the detection chamber; absorbent material located within the waste chamber; and capillary structure located between the detection chamber and the waste chamber.

In another aspect, the present invention provides a detection cartridge that includes a cartridge housing with an interior volume; a sensor operably attached to the cartridge housing, the sensor including a detection surface; a detection chamber located within the interior volume of the cartridge housing, wherein the detection chamber has a volume defined by the detection surface and an opposing surface spaced apart from and facing the detection surface, wherein the opposing surface includes a flow front control feature; a waste chamber located within the interior volume of the cartridge housing, the waste chamber in fluid communication with the detection chamber; one or more sealed modules, wherein each module of the one or more sealed modules includes an exit port attached to the cartridge housing through one or more module ports that open into the interior volume of the cartridge housing. Each module further includes a module housing with an exit port and a sealed interior volume; an exit seal located over the exit port of the module; and a plunger located within the interior volume of the module housing. The plunger is movable from a loaded position in which the plunger is distal from the exit port to an unloaded position in which the plunger is proximate the exit port, and movement of the plunger towards the exit port opens the exit seal such that material from the interior volume of the module housing exits through the exit port into the interior volume of the cartridge housing.

In another aspect, the present invention provides a method of moving sample material through a detection cartridge that includes delivering sample material into the interior volume of the housing of the detection cartridge, wherein the sample material flows into the detection chamber, and wherein flow front progression of the sample material through the detection chamber and towards the waste chamber is controlled at least in part by the flow front control feature on the opposing surface within the detection chamber.

In another aspect, the present invention provides a sealed module including a housing with an exit port and a sealed interior volume; an exit seal located over the exit port; a first chamber located within the interior volume of the housing, the first chamber having a liquid located therein; a second chamber located within the interior volume of the housing, the second chamber including a reagent located therein; an inter-chamber seal isolating the second chamber from the first chamber within the housing; and a plunger, wherein the first chamber, the inter-chamber seal, the second chamber, and the exit seal are located between the plunger and the exit port, and wherein the plunger is movable from a loaded position in which the plunger is distal from the exit port to an unloaded position in which the plunger is proximate the exit port. Movement of the plunger towards the exit port opens the inter-chamber seal such that the liquid in the first chamber contacts the reagent in the second chamber, and wherein further movement of the plunger into the unloaded position opens the exit seal such that the liquid and the reagent from the interior volume of the housing exit through the exit port.

In another aspect, the present invention provides a method of delivering materials using a sealed module of the invention. The method includes moving a plunger towards the exit port of the sealed module to open the inter-chamber seal and force the liquid from the first chamber into contact with the reagent in the second chamber; and moving the plunger towards the exit port to open the exit seal and expel the liquid and the reagent from the interior volume of the housing through the exit port.

In another aspect, the present invention provides a module that includes a housing with an exit port and a sealed interior volume; an exit seal located over the exit port; a chamber located within the interior volume of the housing, the chamber having one or more reagents located therein; a plunger movable from a loaded position in which the plunger is distal from the exit port to an unloaded position in which the plunger is proximate the exit port; and an input port in fluid communication with the chamber, wherein the input port enters the chamber between the plunger and the exit port when the plunger is in the loaded position. Movement of the plunger towards the exit port opens the exit seal such that material from the interior volume of the housing exits through the exit port.

In another aspect, the present invention provides a method of delivering materials using a module of the invention. The method includes delivering sample material comprising a liquid into the chamber of the module through an input port, wherein the sample material contacts the reagent located within the chamber; and moving the plunger towards the exit port to open the exit seal such that the liquid exits from the chamber through the exit port.

These and other features and advantages of the detection systems and methods of the present invention may be described in connection with various illustrative embodiments of the invention below.

### BRIEF DESCRIPTION OF THE DRAWINGS

FIG. 1 is a schematic diagram of one exemplary detection cartridge according to the present invention.

FIG. 2A is a plan view of one exemplary opposing surface including flow front control features according to the present invention.

FIG. 2B is a perspective view of another exemplary opposing surface including flow front control features according to the present invention.

FIG. 2C is a cross-sectional view of another exemplary opposing surface including flow front control features according to the present invention.

FIG. 2D is a cross-sectional view of another exemplary opposing surface including flow front control features according to the present invention.

FIG. 2E is a cross-sectional view of another exemplary opposing surface including flow front control features according to the present invention.

FIG. 2F is a plan view of another exemplary opposing surface including flow front control features according to the present invention.

FIG. 3 is a plan view of an opposing surface including flow control features in the form of hydrophobic and hydrophilic regions.

FIG. 4 is a plan view of another exemplary opposing surface including flow control features according to the present invention.

FIG. 5 is a plan view of another exemplary opposing surface including flow control features according to the present invention.

FIG. 6 is a schematic diagram of one exemplary detection cartridge according to the present invention.

FIG. 6A is an enlarged cross-sectional view of an alternative exemplary opening into a waste chamber in a detection cartridge according to the present invention.

FIG. 6B is an exploded diagram of the components depicted in FIG. 6A.

FIG. 7A depicts one alternative connection between a detection chamber and a waste chamber in a detection cartridge according to the present invention,

FIG. 7B is a cross-sectional view of the flow passage of FIG. 7A taken along line 7B-7B.

FIG. 8A is a cross-sectional diagram of one exemplary module that may be used in connection with the present invention.

FIG. 8B is a cross-sectional diagram of the module of FIG. 8A during use.

FIG. 8C is an enlarged partial cross-sectional view of an alternative plunger and tip seated in the unloaded position within a module of the present invention.

FIG. 8D is a cross-sectional view taken along line 8D-8D in FIG. 8C.

### DETAILED DESCRIPTION OF EXEMPLARY EMBODIMENTS OF THE INVENTION

In the following detailed description of exemplary embodiments of the invention, reference is made to the accompanying figures of the drawings which form a part hereof, and in which are shown, by way of illustration, specific embodiments in which the invention may be practiced. It is to be understood that other embodiments may be utilized and structural changes may be made without departing from the scope of the present invention.

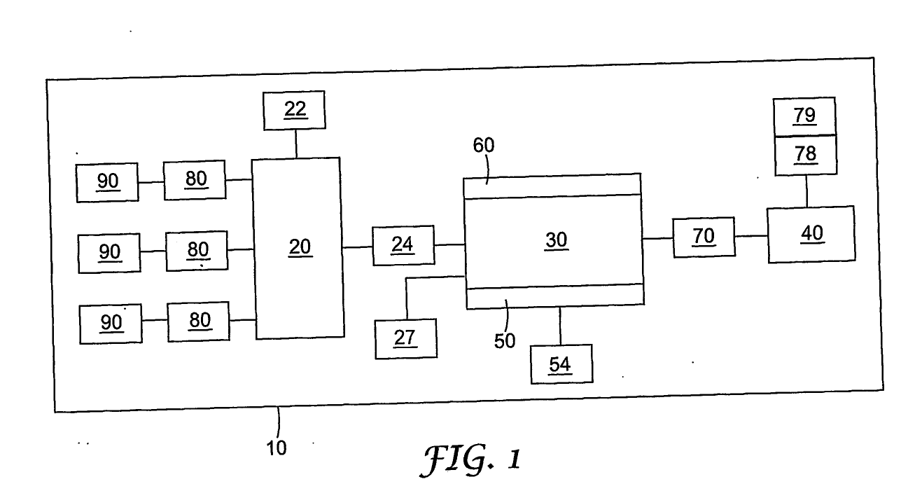

In one aspect, the present invention provides detection cartridges that include an integrated sensor and fluid control features that assist in selective delivery of a sample analyte to the sensor. The exemplary detection cartridge 10 depicted schematically in FIG. 1 includes a staging chamber 20, detection chamber 30, waste chamber 40, sensor 50, volumetric flow control feature 70, and modules 80. In general, the detection cartridge 10 of FIG. 1 may be described as having an interior volume that includes the staging chamber 20, detection chamber 30 and waste chamber 40, with the different chambers defining a downstream flow direction from the staging chamber 20 through the detection chamber 30 and into the waste chamber 40. As a result, the detection chamber 30 may be described as being upstream from the waste chamber 40 and the staging chamber 20 may be described as being upstream from the detection chamber 30. Not every detection cartridge according to the present invention may necessarily include the combination of components contained in detection cartridge 10 of FIG. 1.

The detection chamber 30 of the detection cartridge 10 preferably defines an interior volume between the detection surface of the sensor 50 and an opposing surface 60 located opposite from the detection surface of the sensor. The detection chamber 30 may preferably provide sidewalls or other structures that define the remainder of the interior volume of the detection chamber 30 (i.e., that portion of the detection chamber 30 that is not defined by the detection surface of the sensor 50 and the opposing surface 60).

Also depicted in FIG. 1 is a connector 54 that may preferably be operably connected to sensor 50 to supply, e.g., power to the sensor 50. The connector 50 may preferably supply electrical energy to the sensor 50, although in some embodiments the connector may be used to supply optical energy or any other form of energy required to operate the sensor 50. The connector 54 may also function to connect the sensor 50 to a controller or other system that may supply control signals to the sensor 50 or that may receive signals from the sensor 50. If necessary, the connector 54 (or additional connectors) may be operably connected to other components such as valves, fluid monitors, temperature control elements (to provide heating and/or cooling), temperature sensors, and other devices that may be included as a part of the detection cartridge 10.

In addition to the detection chamber 30, the detection cartridge 10 depicted in FIG. 1 also includes an optional waste chamber 40 into which material flows after leaving the detection chamber 30. The waste chamber 40 may be in fluid communication with the detection chamber 30 through a volumetric flow control feature 70 that can be used to control the rate at which sample material from the detection chamber 30 flows into the waste chamber 40.

The volumetric flow control feature 70 may preferably draw fluid through the detection chamber 30 so that it can move into the waste chamber 40. In various exemplary embodiments as described herein, the volumetric flow control feature 70 may include one or more of the following components: one or more capillary channels, a porous membrane, absorbent material, a vacuum source, etc. These different components may, in various embodiments, limit or increase the flow rate depending on how and where they are deployed within the cartridge 10. For example, a capillary structure may be provided between the detection chamber 30 and the waste chamber 40 to limit flow from the detection chamber 30 into the waste chamber 40 if, e.g., the waste chamber 40 includes absorbent material that might cause excessively high flow rates in the absence of a capillary structure.

Another feature depicted in FIG. 1 is a vent 78 that may preferably be provided to place the interior volume of the detection cartridge 10 in fluid communication with the ambient atmosphere (i.e., the atmosphere in which the detection cartridge 10 is located) when the vent 78 is an open condition. The vent 78 may also preferably have a closed condition in which air flow through the vent 78 is substantially eliminated. Closure of the vent 78 may, in some embodiments, effectively halt or stop fluid flow through the interior volume of the detection cartridge 10. Although depicted as leading into the waste chamber 40, one or more vents may be provided and they may be directly connected to any suitable location within the detection cartridge 10, e.g., staging chamber 20, detection chamber 30, etc. The vent 78 may take any suitable form, e.g., one or more voids, tubes, fitting, etc.

The vent 78 may include a closure element 79 in the form of a seal, cap, valve, or other structure(s) to open, close or adjust the size of the vent opening. In some embodiments, the closure element 79 may be used to either open or close the vent. In other embodiments, the closure element 79 may be adjustable such that the size of the vent opening may be adjusted to at least one size between fully closed and fully open to adjust fluid flow rate through the detection cartridge 10. For example, increasing the size of the vent opening (using, e.g., the closure element 79) may increase fluid flow rate while restricting the size of the vent opening may cause a controllable reduction the fluid flow rate through the interior volume of the detection cartridge 10, e.g., through the staging chamber 20, detection chamber 30, etc. If the vent 78 includes multiple orifices, one or more of the orifices can be opened or closed using the closure element(s), etc.

Although volumetric flow rate of fluid moving through the detection chamber 30 may be controlled by the volumetric flow control feature 70, it may be preferred to provide for control over the flow front progression through the detection chamber 30. Flow front progression control may assist in ensuring that all portions of a detection surface of the sensor 50 exposed within the detection chamber 30 are covered or wetted out by the fluid of the sample analyte such that bubbles or voids are not formed. It may be preferred for example that the flow front progress through the detection chamber 30 in the form of a generally straight line that is oriented perpendicular to the direction of flow through the detection chamber 30.

In the exemplary embodiment depicted in FIG. 1, the flow front control features may preferably be provided in or on the opposing surface 60. This may be particularly true if the sensor 50 relies on physical properties that may be affected by the shape and/or composition of the detection surface, e.g., if the detection surface is part of a sensor that relies on acoustic energy transmission through a waveguide that forms the detection surface or that lies underneath the detection surface. Discontinuities in physical structures or different materials arranged over the detection surface may, e.g., cause the acoustic energy to propagate over the detection surface in a manner that is not conducive to accurate detection of a target analyte within the detection chamber 30. Other sensor technologies, e.g., optical, etc., may also be better-implemented using detection surfaces that do not, themselves, include physical structures or combinations of different materials to control fluid flow front progression within a detection chamber.

In view of the concerns described above, it may be preferred to provide flow front control features in or on the opposing surface 60 of the detection chamber 30 to assist in the control of fluid flow progression over the detection surface of sensor 50. Flow front control may preferably provide control over the progression of sample material over the detection surface while also reducing or preventing bubble formation (or retention) on the detection surface.

The flow front control features provided on the opposing surface 60 may preferably be passive, i.e., they do not require any external input or energy to operate while the fluid is moving through the detection chamber 30. The flow front control features may also preferably operate over a wide range of sample volumes that may pass through the detection chamber 30 (e.g., small sample volumes in the range of 10 microliters or less up to larger sample volumes of 5 milliliters or more).

It may be preferred that the opposing surface 60 and the detection surface of the sensor 50 be spaced apart from each other such that the opposing surface 60 (and any features located thereon) does not contact the detection surface of the sensor 50. With respect to acoustic sensors, even close proximity may adversely affect the properties of the sensor operation. It may be preferred, for example, that spacing between the detection surface of the sensor 50 and the lowermost feature of the opposing surface 60 be 20 micrometers or more, or even more preferably 50 micrometers or more. For effective flow front control, it may be preferred that the distance between the lowermost feature of the opposing surface 60 and the detection surface of the sensor 50 be 10 millimeters, alternatively 1 millimeter or less, in some instances 500 micrometers or less, and in other instances 250 micrometers or less.

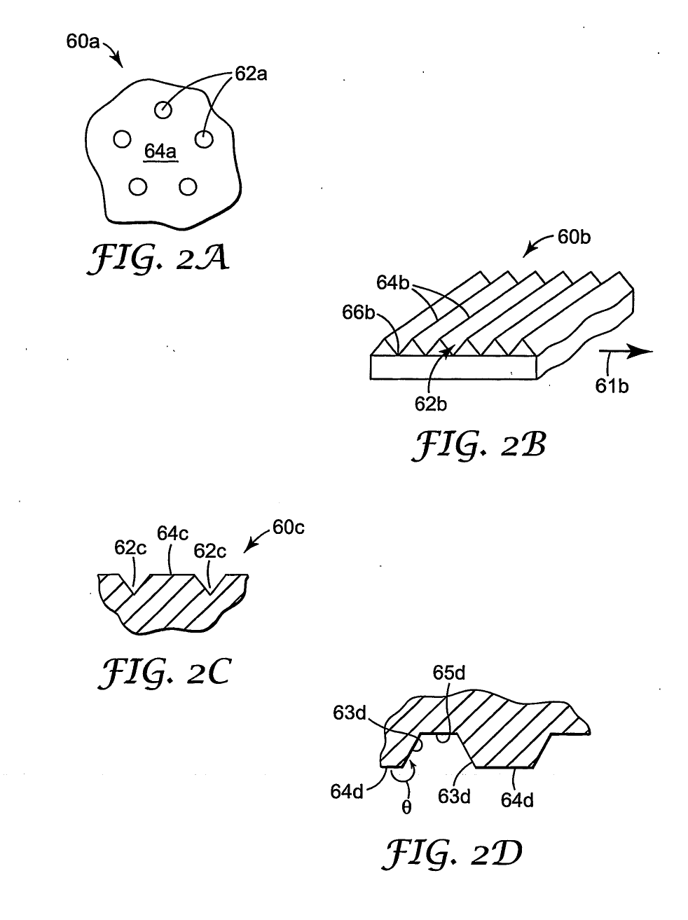

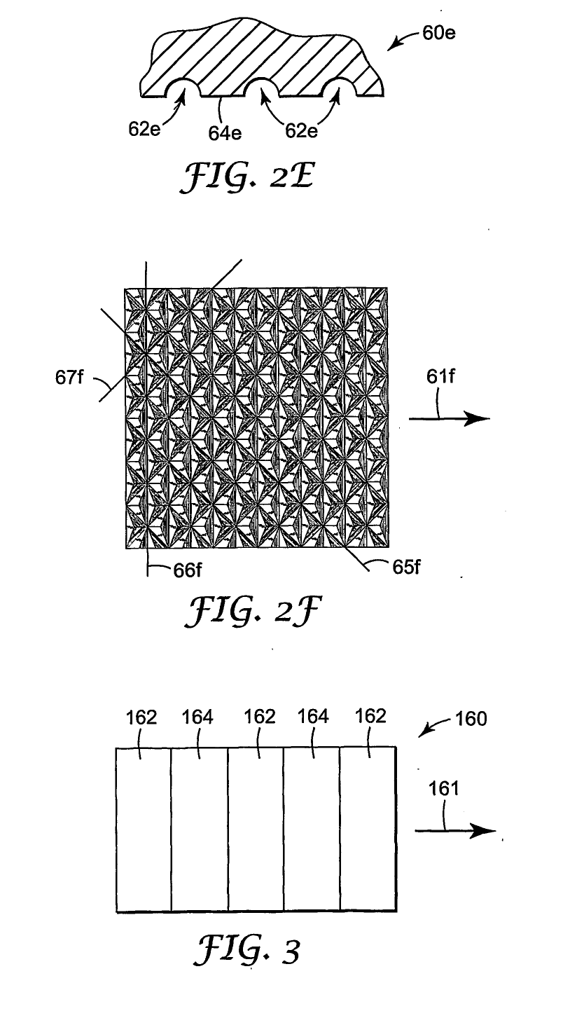

In one class of flow front control features, the opposing surface 60 may include physical structure such as channels, posts, etc. that may be used to control the flow of fluid through the detection chamber 30. Regardless of the particular physical structure, it is preferably of a large enough scale such that flow front progression through the detection chamber is meaningfully affected. FIGS. 2A-2E depict a variety of exemplary physical structures that may be used to control the flow front progression of fluid.

FIG. 2A is a plan view of one type of physical structure on an opposing surface 60a that may provide flow front control. The physical structure includes multiple discrete structures 62a, e.g., posts, embedded or attached beads, etc., dispersed over the opposing surface 60a and protruding from the land area 64a that separates the discrete structures 62a. The discrete structures 62a may be provided in any shape, e.g., circular cylinders, rectangular prisms, triangular prisms, hemispheres, etc. The height, size, spacing, and/or arrangement of the different structures 62a may be selected to provide the desired flow front control depending on fluid viscosity and/or distance between the opposing surface 60a and the detection surface within an detection chamber. It may be preferred that the structures 62a be manufactured of the same material as the land area 64a of the opposing surface 60a between the structures 62a or, alternatively, the structures 62a may be manufactured of one or more materials that differ from the materials that form the land area 64a between structures 62a.

FIG. 2B depicts another exemplary embodiment of physical structure that may be provided in connection with an opposing surface 60b. The physical structure is in the form of triangular channels 62b formed in the opposing surface 60b, with each channel 62b including two peaks 64b on either side of a valley 66b. Although the depicted channels 62b are parallel to each other and extend in a straight line that is perpendicular to the desired fluid flow (see arrow 61b in FIG. 2B), it will be understood that variations in any of these characteristics may be used if they assist in obtaining the desired flow across the detection surface. The channels 62b maybe irregularly sized, irregularly shaped, irregularly spaced, straight, curved, oriented at other than a ninety degree angle to fluid flow, etc. For example, adjacent channels 62b may be immediately adjacent each other as seen in FIG. 2B. Also, although the channels 62b have a triangular cross-sectional shape, channels used in connection with the present invention may have any cross-sectional shape, e.g., arcuate, rectangular, trapezoidal, hemispherical, etc. and combinations thereof.

In other embodiments, the channels may be separated by land areas between peaks or include valleys that have a land area (i.e., that does not reach a bottom and then immediately turn upward to the adjacent peak). The land areas may be flat or take other shapes as desired. One such variation is depicted in FIG. 2C in which channels 62c in opposing surface 60c are provided with land areas 64c separating the channels 62c on opposing surface 60c.

FIG. 2D depicts another variation in physical structures that may be used for flow front control on an opposing surface 60d. The physical structures are provided in the form of channels 62d. The channels 62d of opposing surface 60d have a different shape, i.e., are more rectangular or trapezoidal, including walls 63d and roof 65d, as opposed to the triangular channels of FIGS. 2B and 2C.

Even though the channels 62d are more rectangular in shape, it may be preferred that the wall 63d at the leading edge of each channel 62d forms an angle θ (theta) with the surface 64d leading up to the channel 62d that is less than 270 degrees. As used herein, the “leading edge” of a channel is that edge that is encountered first by liquids moving in the downstream direction over the detection surface. Limiting the angle θ (theta) may promote fluid flow into the channels 62d because higher angles between the walls 63d at the leading edges and the surfaces 64d may impede fluid flow front progression. By virtue of their triangular shape, the channels in the opposing surfaces in FIGS. 2B & 2C inherently possess angles that are conducive to fluid flow into the channels.

FIG. 2E depicts another embodiment of an opposing surface 60e that includes channels 62e with an arcuate (e.g., hemispherical) profile that also provide entrance angles of less than 270 degrees to also preferably promote fluid flow into the channels 62e. The channels 62e may preferably be separated by land areas 64e as depicted in FIG. 2E.

In addition to the variations described above with respect to FIGS. 2A-2E, another variation may be that channels of two or more different shapes may be provided on a single opposing surface, e.g., a mix of triangular, rectangular, hemispherical, etc. channels may be provided on the same opposing surface.

FIG. 2F depicts yet another variation of an opposing surface 60f that includes physical structure to control a fluid flow front within a detection chamber. The depicted surface 60f includes a discrete structures made by a series of triangular-shaped channels formed in the surface 60f along and/or parallel to axes 65f, 66f and 67f. It may be preferred that at least one of the sets of channels be formed in a direction that is generally perpendicular to fluid flow direction as represented by arrow 61f as, for example, the channels along and/or parallel to axis 66f. Together with the angled channels along axes 65f and 67f, perpendicular channels along/parallel to axis 66f form faces on each of the pyramidal structures. Although the depicted pyramid structures have triangular bases, pyramid-shaped structures could be provided with four or more faces if so desired.

Referring again to FIG. 1, flow front control through the detection chamber 30 may also be accomplished without the use of physical structures. In some embodiments, flow front control may be accomplished through the use of hydrophilic and/or hydrophobic materials located on the opposing surface 60. FIG. 3 is a plan view of an opposing surface 160 that includes regions 162 of hydrophobic materials and regions 164 of hydrophilic materials occupying portions of the opposing surface 160. The regions 162 and 164 may preferably be provided as successive bands oriented generally perpendicular to the direction of flow through the detection chamber as illustrated by arrow 161, i.e., from an input end to an output end of a detection chamber (although other hydrophilic/hydrophobic patterns may be used). The hydrophilic and/or hydrophobic materials used in regions 162 and/or 164 may be coated or otherwise provided on the opposing surface 160. In some instances, the material used to construct the opposing surface 160 may itself be considered hydrophilic while a more hydrophobic material is located on selected portions of the opposing surface 160 (or vice versa, i.e., the material used to construct the opposing surface 160 may be hydrophobic and regions of that surface may be coated or otherwise treated to provide hydrophilic regions on the opposing surface).

Generally, the susceptibility of a solid surface to be wet out by a liquid is characterized by the contact angle that the liquid makes with the solid surface after being deposited on the horizontally disposed surface and allowed to stabilize thereon. It is sometimes referred to as the “static equilibrium contact angle,” sometimes referred to herein merely as “contact angle”. As discussed in U.S. Pat. No. 6,372,954 B1 (Johnston et al.) and International Publication No. WO 99/09923 (Johnston et al.), the contact angle is the angle between a line tangent to the surface of a bead of liquid on a surface at its point of contact to the surface and the plane of the surface. A bead of liquid whose tangent was perpendicular to the plane of the surface would have a contact angle of 90 degrees. Typically, if the contact angle is 90 degrees or less, the solid surface is considered to be wet by the liquid. Liquid sample materials that yield a contact angle of near zero on a surface are considered to completely wet out the surface.

Frequently, horizontal surfaces on which drops of water at 20 degrees Celsius exhibit a contact angle of 90 degrees or less are considered to be hydrophilic while horizontal surfaces on which drops of water at 20 degrees Celsius exhibit a contact angle of more than 90 degrees are considered to be hydrophobic.

For the purposes of the present invention, it may be preferred that the hydrophilicity/hydrophobicity of surfaces be determined on a relative scale. For example, it may be preferred that the difference in contact angle between what would be considered hydrophilic and hydrophobic horizontal surfaces be about 10 degrees or more (or, in some instances, 20 degrees or more) for drops of water at 20 degrees Celsius. In other words, the hydrophobic surfaces of the present invention may exhibit a contact angle that is 10 degrees or more (or 20 degrees or more) higher than the contact angle of a hydrophilic surface (for water on a horizontal surface at 20 degrees Celsius).

As used herein, “hydrophilic” is used only to refer to the surface characteristics of a material, i.e., that it is wet by aqueous solutions, and does not express whether or not the material absorbs or adsorbs aqueous solutions. Accordingly, a material may be referred to as hydrophilic whether or not a layer of the material is impermeable or permeable to water or aqueous solutions.

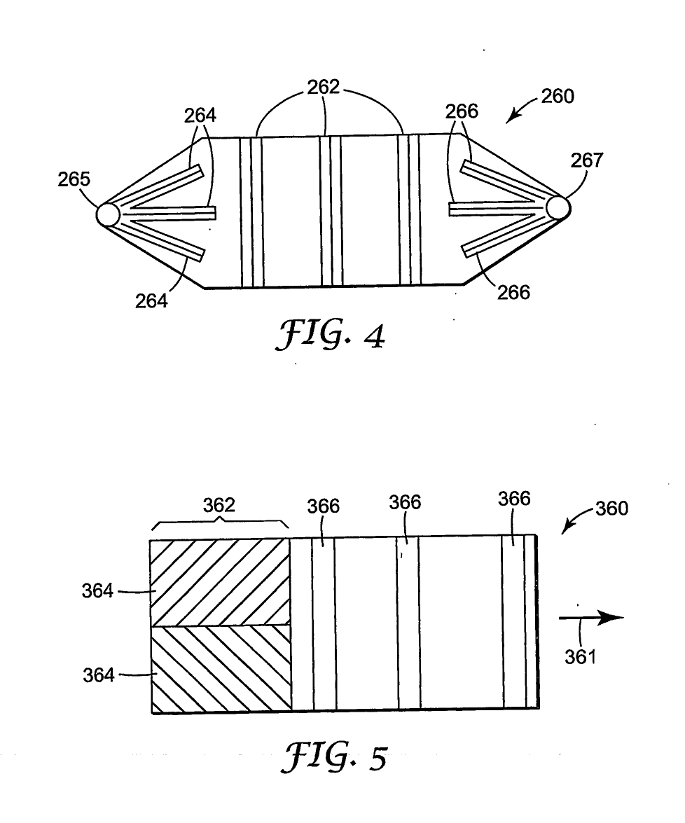

FIG. 4 is a plan view of another embodiment of an opposing surface 260 that may be used in a detection chamber of the present invention. The opposing surface 260 includes physical structures 262 in the form of straight channels that are preferably oriented generally perpendicular to the direction of flow through the detection chamber. In addition to the cross-chamber channels 262, the opposing surface 260 also includes flow directors 264 diverging outwardly towards the sides of the opposing surface 260 in a fan-shaped pattern at the inlet end 265. The opposing surface 260 depicted in FIG. 4 also includes flow directors 266 converging inwardly towards the center of the width of the width of the opposing surface 260 at the outlet end 267 of the opposing surface 260.

In use, the flow directors 264 at the inlet end 265 may preferably assist in expanding the flow front across the width of the opposing surface 260 (and, thus, the detection chamber in which the opposing surface 260 is located) as fluid enters the detection chamber. As the fluid reaches the first cross-chamber channel 262, the flow front may preferably stop moving in the direction of outlet end 267 until the flow front extends across the width the opposing surface 260. Once the flow front reaches across the opposing surface 260, it may preferably advance to the next cross-chamber channel 262 where it again halts until the flow front extends across the width of the opposing surface 260.

The flow front proceeds in the manner described in the preceding paragraph until reaching the optional flow directors 266 near the outlet end of the opposing surface 260. There the flow is directed to the outlet end 267 of the detection chamber where it can be directed to the waste chamber as described herein.

The flow control features depicted in FIG. 5 include an opposing surface 360 that includes an entry section 362 in which a series of channels 364 are oriented at an angle that is not perpendicular to the direction of fluid flow (as indicated by arrow 361). It may be preferred that the channels 364 diverge from a central axis 363 that generally bisects the width of the opposing surface 36Q (where the width is measured generally perpendicular to the flow direction 361) and be arranged in a general V-shape with the width of the V-shape increasing along the flow direction. The channels 366 in second section of the opposing surface 360 may preferably be oriented generally perpendicular the fluid flow direction. Such an arrangement may be beneficial in ensuring fluid flow to the sides of the surface 338 and may also shunt or direct bubbles to the edges of the detection chamber where they may not interfere with operation of the detection surface.

The variety of flow front control approaches described herein may be used in combinations that are not explicitly depicted. For example, it may be preferred to use selected areas of hydrophobic and/or hydrophilic materials on the opposing surface in combination with physical structures (e.g., channels, discrete protruding structures, etc.) to provide control over the flow front progression through a detection chamber in the present invention. Further, although the interior volume of the detection chamber 30 may preferably have a generally rectilinear shape, it will be understood that detection chambers used in connection with the present invention may take other shapes, e.g., cylindrical, arcuate, etc.

Returning to FIG. 1, the optional staging chamber 20 that may also be included within the detection cartridge 10 may be used to stage, mix or otherwise hold sample material before its introduction to the detection chamber 30. The staging chamber 20 may take any suitable form. In some instances, it may be preferred that the volume of the staging chamber 20 be located above (relative to gravitational forces) the detection chamber 30 during use of the cartridge 10 such that static head can be developed within the sample material in the staging chamber 20 that can assist its passive delivery to the detection chamber 30 from the staging chamber 20.

An optional port 22 may be provided in the staging chamber 20 (or in another location that leads to the interior of the cartridge 10) such that material may be introduced into the interior volume of the cartridge 10 by, e.g., by syringe, pipette, etc. If provided, the port 22 may be sealed by, e.g., a septum, a valve, and/or other structure before and/or after materials are inserted into the cartridge 10. In some embodiments, the port 22 may preferably include, e.g., an external structure designed to mate with a test sample delivery device, e.g., a Luer lock fitting, threaded fitting, etc. Although only one port 22 is depicted, it should be understood that two or more separate ports may be provided.

In some embodiments, the staging chamber 20 may be isolated from direct fluid communication with the detection chamber 30 by a flow control structure/mechanism 24 (e.g., a valve). If a flow control structure/mechanism 24 is provided to isolate the detection chamber 30 from the staging chamber 20, then the staging chamber 20 may potentially be more effectively used to store materials before releasing them into the detection chamber 30. In the absence of a flow control structure/mechanism 24, some control over the flow of materials into the detection chamber 30 may potentially be obtained by other techniques, e.g., holding the cartridge 10 in an orientation in which the force of gravity, centripetal forces, etc. may help to retain materials in the staging chamber 20 until their delivery to the detection chamber 30 is desired.

Another optional feature depicted in FIG. 1 is the inclusion of a fluid monitor 27. The fluid monitor 27 may preferably provide for active, real-time monitoring of fluid presence, flow velocity, flow rate, etc. The fluid monitor 27 may take any suitable form, e.g., electrodes exposed to the fluid and monitored using e.g., alternating currents to determine flow characteristics and/or the presence of fluid on the monitors electrodes. Another alternative may involve a capacitance based fluid monitor that need not necessarily be in contact with the fluid being monitored.

Although depicted as monitoring the detection chamber 30, it should be understood that the fluid monitor may be located at any suitable location within the interior volume of the detection cartridge 10. For example, the fluid monitor could be located in the staging chamber 20, the waste chamber 40, etc. In addition, multiple fluid monitors may be employed at different locations within the cartridge 10.

Potential advantages of the fluid monitor 27 may include, e.g., the ability to automatically activate the introduction of sample materials, reagents, wash buffers, etc. in response to conditions sensed by the fluid monitor 27 that are employed in a feedback loop to, e.g., operate actuators 90 associated with modules 80, etc. Alternatively, the conditions sensed by the fluid monitor 27 can provide signals or feedback to a human operator for evaluation and/or action. For some applications, e.g., diagnostic healthcare applications, the fluid monitor 27 may be used to ensure that the detection cartridge is operating properly, i.e., receiving fluid within acceptable parameters.

Also depicted in FIG. 1 are optional modules 80 that may preferably be used to introduce or deliver materials into the cartridge 10 in addition to or in place of ports 22. It may be preferred, as depicted, that the modules 80 deliver materials into the staging chamber 20, although in some instances, they could potentially deliver materials directly into the detection chamber 30. The modules 80 may be used to deliver a wide variety of materials, although it may be preferred that the delivered materials include at least one liquid component to assist in movement of the materials from the module 80 and into the cartridge 10. Among the materials that could be introduced using modules 80 are, e.g., sample materials, reagents, buffers, wash materials, etc. Control over the introduction of materials from the modules 80 into the cartridge 10 maybe obtained in a number of manners, e.g., the modules 80 may be isolated from the cartridge 10 by a seal, valve, etc. that can be opened to permit materials in the modules 80 to enter the cartridge 10.

It may be preferred that the modules 80 be independent of each other such that the materials contained within each module 80 can be introduced into the detection cartridge at selected times, at selected rates, in selected orders, etc. In some instances an actuator 90 may be associated with each module 80 to move the materials within the module 80 into the cartridge 10. The actuators 90 may be selected based on the design of the module 80. The actuators 90 may be manually operated or they may be automated using, e.g., hydraulics, pneumatics, solenoids, stepper motors, etc.

A potential advantage of using modules 80 to deliver materials such as reagents, buffers, etc. may be the opportunity to tailor the cartridge 10 for use with a wide variety of sample materials, tests, etc.

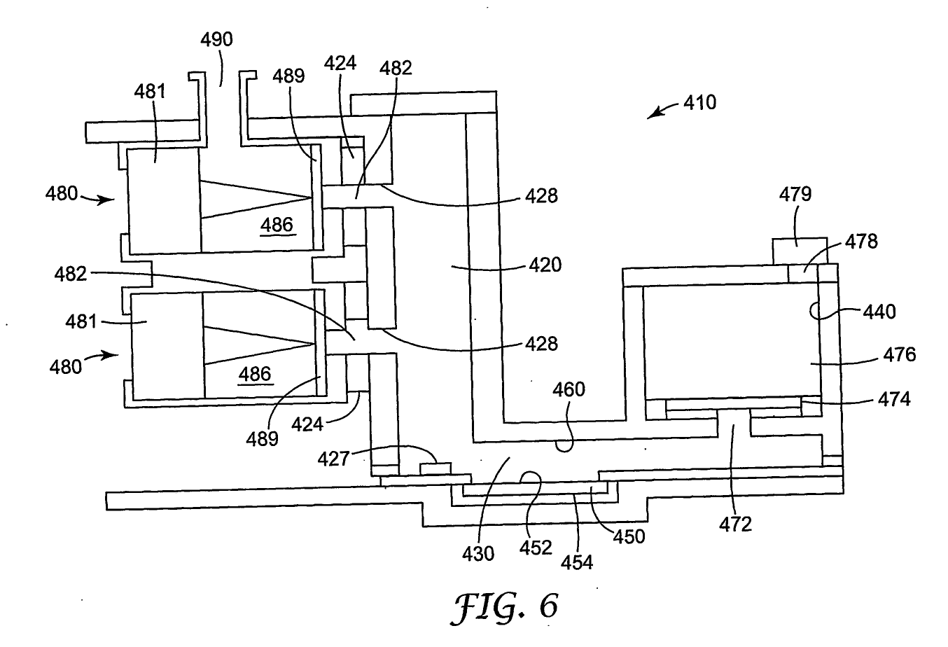

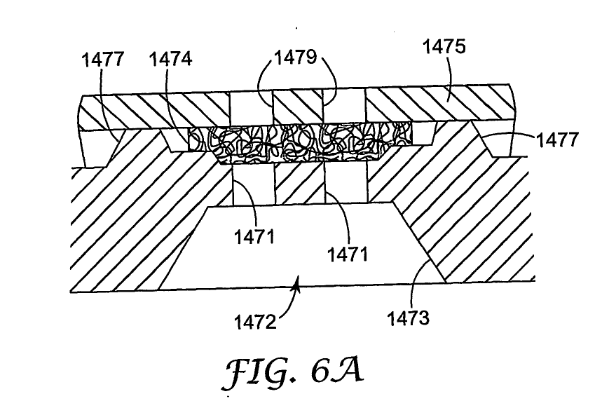

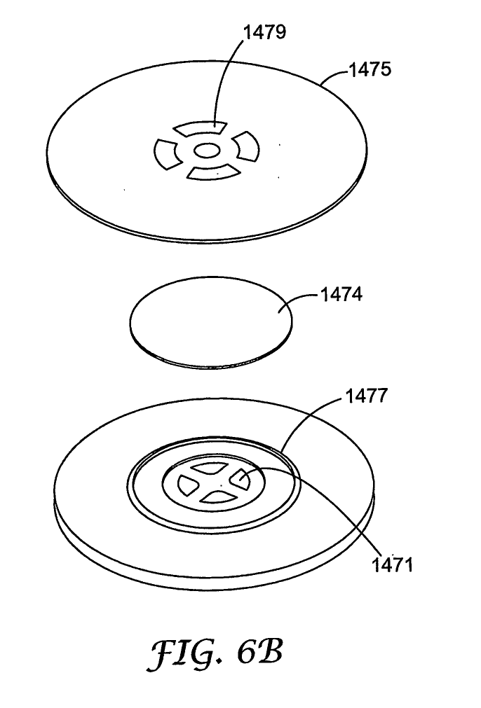

Various aspects of the detection cartridge 10 schematically depicted in FIG. 1 having been thus described, one exemplary embodiment of a detection cartridge 410 including a staging chamber 420, detection chamber 430 and waste chamber 440 is depicted in FIG. 6. The detection cartridge 410 includes a housing 412 and a sensor 450 having a detection surface 452 exposed within the detection chamber 430.

It may be preferred that the sensor 450 be an acousto-mechanical sensor such as, e.g., a Love wave shear horizontal surface acoustic wave sensor. As depicted, the sensor 450 may preferably be attached such that, with the possible exception of its perimeter, the backside 454 of the sensor 450 (i.e., the surface facing away from the detection chamber 430) does not contact any other structures within the cartridge 410. Examples of some potentially suitable methods of attaching acousto-mechanical sensors within a cartridge that may be used in connection with the present invention may be found in, e.g., U.S. patent application Ser. No. 60/533,176, filed on Dec. 30, 2003 as well as PCT Application No. ______, titled “Surface Acoustic Wave Sensor Assemblies”, filed on even date herewith, (Attorney Docket No. 58928WO003).

It should, however, be understood that acousto-mechanical sensors represent only one class of sensors that may be used in connection with the present invention. Many other sensor technologies may be used in connection with the cartridges of the present invention, e.g., surface plasmon resonance, electrochemical detection, conductivity sensors, fluorescent microarrays, chemiluminescence, etc.

Regardless of the specific detection technology used in sensor 450, it may be preferred that the portion of the detection surface 452 exposed within the detection chamber 430 be positioned to contact sample material flowing through the detection chamber 430. It may be preferred, for example, that the detection surface 452 be located at the bottom (relative to gravitational forces) of the detection chamber 430 such that materials flowing through the detection chamber 430 are urged in the direction of the detection surface 452 through at least the force of gravity (if not through other forces).

The detection chamber 430 may also preferably include an opposing surface 460 spaced apart from and facing the detection surface 452. One or more different flow front control features may preferably be provided on the opposing surface 460 to assist in controlling the progression of a flow front through the detection chamber 430. Various examples of potentially suitable flow front control features are discussed herein.

It may be preferred that the opposing surface 460 and the detection surface 452 be spaced apart from each other such that the opposing surface 460 (and any features located thereon) does not contact the detection surface 452. With respect to acoustic sensors, even close proximity may adversely affect the properties of the sensor operation if the opposing surface 460 disrupts the propagation of acoustic energy by the detection surface 452. It may be preferred, for example, that spacing between the detection surface 452 and the lowermost feature of the opposing surface 460 facing the active part of the detection surface 452 be 20 micrometers or more, or even more preferably 50 micrometers or more. For effective flow front control, it may be preferred that the distance between the lowermost feature of the opposing surface 460 and the detection surface 452 be 10 millimeters, alternatively 1 millimeter or less, in some instances 500 micrometers or less, and in other instances 250 micrometers or less.

The cartridge 410 of FIG. 6 also includes a waste chamber 440 that is in fluid communication with the detection chamber 430 and into which sample material flows after leaving the detection chamber 430. The cartridge 410 may preferably include a volumetric flow control feature interposed in the fluid path between the detection chamber 430 and the waste chamber 440. The volumetric flow control feature may preferably function to control the rate at which sample material from the detection chamber 430 flows into the waste chamber 440.

Although the volumetric flow control feature may take many different forms, in the embodiment depicted in FIG. 6 it is provided in the form of an opening 472 over which a capillary structure in the form of a porous membrane 474 is located. In addition to the porous membrane 474, a mass of absorbent material 476 is located within the waste chamber 440.

The porous membrane 474 may preferably provide a fluid pressure drop from the side facing the detection chamber 430 to the side facing the waste chamber 440. The porous membrane 474 preferably assists in controlling the flow rate from the detection chamber 430 into the waste chamber 440. The pressure drop may preferably be provided by capillary action of the passageways within the porous membrane 474. The pressure drop across a porous membrane is typically a function of the pore size and the thickness of the membrane. It may be preferred that the porous membrane have a pore size in the range of, e.g., 0.2 microns to 50 microns. Some suitable examples of materials that may be useful as a porous membrane include, e.g., acrylic copolymers, nitrocellulose, polyvinylidene fluoride (PVDF), polysulfone, polyethersulfone, nylon, polycarbonate, polyester, etc.

Referring to FIGS. 6A & 6B, an alternative structure using a porous membrane 1474 to control fluid flow rate into a waste chamber is depicted. The opening 1472 includes a series of orifices 1471 formed through the material of the housing. The opening 1472 may preferably include a chamfer 1473 to preferably assist in fluid flow through the opening 1472 by avoiding a sharp edge that may inhibit flow into and through the opening 1472 (alternatively, radiused, rounded or smoothed edges, etc. could be used).

The porous membrane 1474 is held in place by a cover plate 1475 that, in the preferred embodiment may be ultrasonically welded over the orifices 1471 with the porous membrane 1474 located therebetween. The cover plate 1475 may preferably include orifices 1479 through which fluids may pass into a waste chamber. The ultrasonic welding of the cover plate 1475 may be assisted by the use of an energy director 1477 surrounding the opening 1472 and the height of the energy director 1477 may be sufficient to allow some clearance for the thickness of the porous membrane 1474. In such a system, the cover plate 1475 and energy director 1477 may assist in the formation of a fluid-tight attachment without destruction of the porous membrane 1474. Other techniques for retaining the membrane 1474 over opening 1472 may also be used, e.g., adhesives, thermal welding, solvent welding, mechanical clamping, etc. These techniques may be used with or without a cover plate 1475, i.e., the porous membrane 1474 itself may be directly attached to the structures surrounding the opening 1472.

Referring again to the embodiment of FIG. 6, although the membrane 474 may draw fluid from the detection chamber 430, surface tension in the fluid may prevent the fluid from flowing out of the membrane 474 and into the waste chamber 440. As a result, it may be preferred to draw fluid from the membrane 474 into the waste chamber 40 using, e.g., negative fluid pressure within the waste chamber 440. The negative fluid pressure within the waste chamber 440 may be provided using a variety of techniques. One technique for providing a negative fluid pressure within the waste chamber 440 may include, e.g., absorbent material 476 located within the waste chamber 440 as depicted in FIG. 6. One alternative technique for providing a negative fluid pressure within the waste chamber 440 is a vacuum within the waste chamber 440. Other alternative techniques may also be used.

It may be preferred that negative fluid pressure within the waste chamber 440 be provided passively, e.g., through the use of absorbent material or other techniques that do not require the input of energy (as would, for example, maintaining a vacuum within the waste chamber). Examples of some potentially suitable absorbent materials that may provided within the waste chamber 440 may include, but are not limited to, foams (e.g., polyurethane, etc.), particulate materials (e.g., alumina-silicate, polyacrylic acid, etc.), granular materials (e.g., cellulose, wood pulp, etc.).

If the waste chamber 440 is provided with absorbent material 476 located therein as depicted in FIG. 6, it may be preferred that the absorbent material be in physical contact with the side of the membrane 474 (or any orifices 1479 in a cover plate 1475 as seen in FIGS. 6A & 6B) facing the interior of the waste chamber 440. A gap between the absorbent material 476 and the membrane 474 may limit or prevent fluids from leaving the membrane 474 and entering the waste chamber 440 because of, e.g., surface tension within the fluid as contained in the membrane 474.

If absorbent material 476 is provided within the waste chamber 440, it may be beneficial to provide a variety of layers of absorbent materials to control the volumetric flow rate into the waste chamber 440. For example, a first layer of absorbent material may be provided proximate the membrane 474, with the first layer material having a characteristic wicking rate and a defined fluid volume. After the first layer of absorbent material has been loaded to its capacity, the fluid entering the waste chamber 440 may be drawn into a second layer of absorbent material with a different wicking rate, thereby potentially providing a different negative pressure in the waste chamber 440.

Changing the negative pressure within the waste chamber 440 using, e.g., different layers of absorbent materials, may be used to compensate for other changes within the cartridge 410 such as, e.g., changes in fluid head pressure as sample material is drawn through the cartridge 410. Other techniques may also be used to compensate for changes in the fluid head pressure such as, e.g., changing a vacuum level held in the waste chamber, opening one or more vents in the cartridge, etc.

The embodiment of FIG. 6 includes a vent 478 in the waste chamber 440 that may place the interior volume of the waste chamber 440 in fluid communication with ambient atmosphere. Opening and/or closing the vent 478 may be used to control fluid flow into the waste chamber 440 and, thus, through the cartridge 410. Furthermore, the vent 478 may be used to reduce pressure within the waste chamber 440 by, e.g., drawing a vacuum, etc. through the vent 478.

Although depicted as being in direct fluid communication with the waste chamber 440, one or more vents may be provided and they may be directly connected to any suitable location that leads to the interior volume of the detection cartridge 410, e.g., staging chamber 420, detection chamber 430, etc. The vent 478 may take any suitable form, e.g., one or more voids, tubes, fitting, etc.

The vent 478 may preferably include a closure element 479 in the form of a seal, cap, valve, or other structure(s) to open, close or adjust the size of the vent opening. If provided as a seal, the seal may be adhesively or otherwise attached over or located within the vent 478. In some embodiments, the closure element 479 may be used to either open or close the vent. In other embodiments, the closure element 479 may be adjustable such that the size of the vent opening may be adjusted to at least one size between fully closed and fully open to adjust fluid flow rate through the detection cartridge 410. For example, increasing the size of the vent opening may increase fluid flow rate while restricting the size of the vent opening may cause a controllable reduction the fluid flow rate through the interior volume of the detection cartridge 410, e.g., through the staging chamber 420, detection chamber 430, etc. If the vent 478 includes multiple orifices, one or more of the orifices can be opened or closed to control fluid flow, etc.

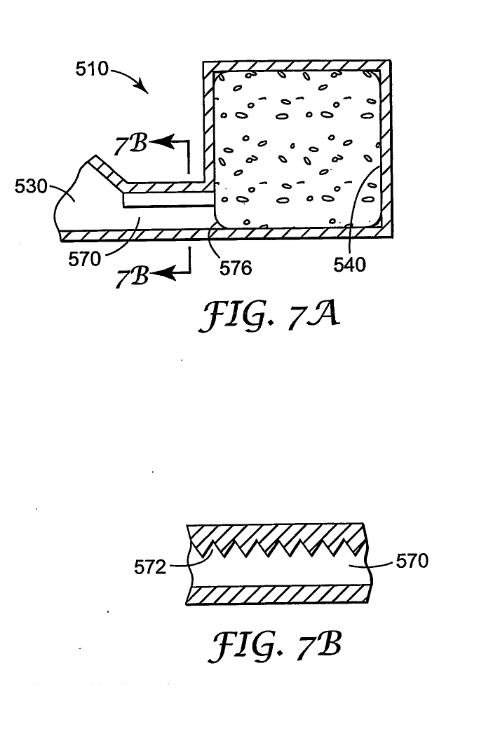

FIGS. 7A & 7B depict a portion of an alternative cartridge 510 including a portion of a detection chamber 530 and a waste chamber 540. The waste chamber 540 and the detection chamber 530 are, in the depicted embodiment, separated by a capillary structure in the form of a flow passage 570 that includes a set of capillary channels 572 that may preferably draw fluid from the detection chamber 530 by capillary forces. The particular shape of the capillary channels 572 may be different from those depicted in the cross-sectional view of FIG. 7B. Also, the number of capillary channels 572 provided in the flow passage may vary from as few as one capillary channel to any selected number of multiple capillary channels.

In the embodiment of FIGS. 7A & 7B, the flow passage 570 may preferably take the place of the porous membrane used in connection with the embodiment of FIG. 6. The capillary channel or channels 570 preferably provide the desired level of negative fluid pressure to draw fluid from the detection chamber 530.

In some instances, it may be preferred to provide both a porous membrane and one or more capillary channels to provide a capillary structure between the detection chamber and the waste chamber in detection cartridges of the present invention. Other capillary structures such as tubes, etc. could be substituted for the exemplary embodiments described herein.

Although the capillary channels 572 may draw fluid from the detection chamber 530, surface tension in the fluid may prevent the fluid from flowing out of the flow passage 570 and into the waste chamber. 540. As a result, it may be preferred to draw fluid from the flow passage 570 into the waste chamber 540 using, e.g., negative fluid pressure within the waste chamber 540. The negative fluid pressure within the waste chamber 540 may be provided using a variety of techniques. One technique for providing a negative fluid pressure within the waste chamber 540 may include, e.g., absorbent material 576 located within the waste chamber 540 as depicted in FIG. 7A. One alternative technique for providing a negative fluid pressure within the waste chamber 540 is a vacuum within the waste chamber 540. Other alternative techniques may also be used.

It may be preferred that negative fluid pressure within the waste chamber 540 be provided passively, e.g., through the use of absorbent material or other techniques that do not require the input of energy (as would, for example, maintaining a vacuum within the waste chamber). The use of absorbent materials within a waste chamber is described above in connection with the embodiment depicted in FIG. 6.

If absorbent materials are used within the waste chamber 540, it may be preferred that the absorbent material be in contact with the end or ends of any capillary channel(s) 572 to overcome any surface tension that might otherwise prevent fluid from exiting the capillary channel(s).

Referring again to the cartridge depicted in FIG. 6, the staging chamber 420 may be provided upstream from the detection chamber 430. The staging chamber 420 may provide a volume into which various components may be introduced before entering the detection chamber 430. Although not depicted, it should be understood that the staging chamber 420 could include a variety of features such as, e.g., one or more reagents located therein (e.g., dried down or otherwise contained for selective release at an appropriate time); coatings (e.g., hydrophilic, hydrophobic, etc.); structures/shapes (that may, e.g., reduce/prevent bubble formation, improve/cause mixing, etc.).

Also, the fluid path between the staging chamber 420 and the detection chamber 430 may be open as depicted in FIG. 6. Alternatively, the fluid path between the staging chamber 420 and the detection chamber 430 may include a variety features that may perform one or more functions such as, e.g., filtration (using, e.g., porous membranes, size exclusion structures, beads, etc.), flow control (using, e.g., one or more valves, porous membranes, capillary tubes or channels, flow restrictors, etc.), coatings (e.g., hydrophilic, hydrophobic, etc.), structures/shapes (that may, e.g., reduce/prevent bubble formation and/or transfer, improve mixing, etc.).

Another optional feature depicted in FIG. 6 is the inclusion of a fluid monitor 427 in the flow path between the staging chamber 420 and the detection chamber 430. The fluid monitor 427 may preferably provide for active, real-time monitoring of fluid presence, flow velocity, flow rate, etc. The fluid monitor 427 may take any suitable form, e.g., electrodes exposed to the fluid and monitored using e.g., alternating currents to determine flow characteristics and/or the presence of fluid on the monitors electrodes. Another alternative may involve a capacitance based fluid monitor that need not necessarily be in contact with the fluid being monitored.

Potential advantages of the fluid monitor 427 may include, e.g., the ability to automatically activate the introduction of sample materials, reagents, wash buffers, etc. in response to conditions sensed by the fluid monitor 427. Alternatively, the conditions sensed by the fluid monitor 427 can provide signals or feedback to a human operator for evaluation and/or action. For some applications, e.g., diagnostic healthcare applications, the fluid monitor 427 may be used to ensure that the detection cartridge is operating properly, i.e., receiving fluid within acceptable parameters.

The exemplary cartridge 410 depicted in FIG. 6 includes two modules 480 arranged to deliver material into the staging chamber 420 of the cartridge 410 (it should be understood that the orientation or direction of the modules 480 with respect to the staging chamber 420 may vary from that depicted). The modules 480 deliver their materials into the staging chamber 420 through module ports 428 that open into the staging chamber 420. The modules 480 may preferably be attached to the module ports 428 by an adhesive 424 or other material capable of providing a suitable fluid-tight seal between the modules 480 and the module ports 428. Any suitable technique for attaching the modules 480 to the module ports 428 may be substituted for the adhesive 424. In some instances, the modules 480 may be welded (chemically, thermally, ultrasonically, etc.) or otherwise attached over the module ports 428. In other instances, the modules 480 may be connected to the module ports using complementary structures such as threaded fittings, Luer locks, etc.

Although other exemplary embodiments of modules that may be used to introduce materials into the cartridge 410 are described elsewhere, each of the modules 480 depicted in FIG. 6 includes a seal 489 over an opening 482 that is aligned over the module port 428 leading into staging chamber 420. Each of the modules 480 also includes a plunger 481 that defines a chamber 486 located between the seal 489 and the plunger 481. The material or materials to be delivered into the staging chamber 420 are typically located within the chamber 486 before the plunger 481 is used to deliver the contents of the module 480 into the staging chamber 420.

In the depicted embodiment, the plunger 481 may preferably be designed to pierce, tear or otherwise open the seal 489 to allow the materials with the modules 480 to enter the staging chamber 420. The depicted plungers 481 include piercing tips for that purpose. It should be understood that the modules 480 could be isolated from the staging chamber 420 by valves or any other suitable fluid structure used to control movement of materials between chambers.

One variation depicted in PIG. 6 is that the upper module 480 includes a port 490 opening into the chamber 486 of the module 480. The port 490 may be used to deliver materials into the chamber 486 for subsequent delivery to the staging chamber using the module 480. For example, the port 490 may be used to introduce a collected specimen, etc. into the module 480 where it can then be introduced into the staging chamber 420 at selected times and/or rates. In addition, the chamber 486 of the module 480 receiving the sample material may include one or more reagents or other materials that may contact the sample material upon its introduction to the module 480. Although not depicted, it may be preferred that the port 490 be sealed before and/or after sample material is introduced into the module 480 using a valve or other structures/materials. The port 490 may be sealed by, e.g., a septum, a valve, induction welded seal, cap, and/or other structure before and/or after materials are inserted into the module 480.

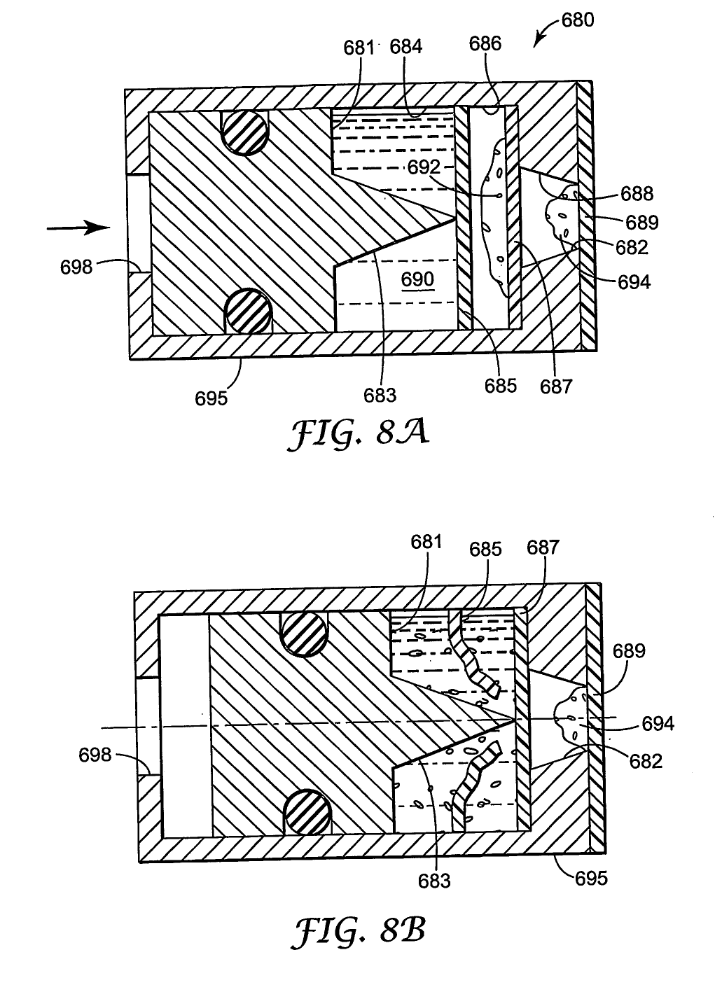

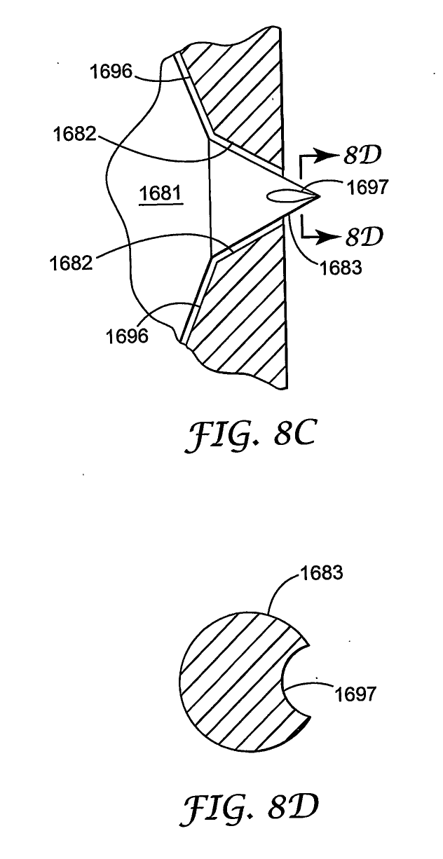

One exemplary embodiment of a module 680 that may be used to deliver reagents and/or other materials in accordance with the present invention is depicted in the cross-sectional views of FIGS. 8A & 8B. The depicted exemplary module 680 includes multiple chambers, each of which may contain the same or different materials and each of which may preferably be hermetically sealed from each other. It may be preferred that the module 680 be designed such that the materials within the different chambers mix as they are introduced to each other.

By storing the different materials within separate chambers, it may be possible to provide materials in the module 680 that are preferably not mixed until needed. For example, some substances may preferably be stored in a dry state to, e.g., prolong their shelf life, usable life, etc., but the same substances may need to be mixed in liquids that may include water, etc. to provide a usable product. By providing the ability to mix and/or dispense these materials on demand, the modules of the present invention can provide a convenient storage and introduction device for many different materials.

The depicted module 680 includes three chambers 684, 686 and 688 within housing 695. The chambers may preferably be separated by a seal 685 (located between chambers 684 and 686) and seal 687 (located between chambers 686 and 688). The depicted module 680 also includes plunger 681 with a tip 683 that, in the depicted embodiment, is designed to pierce seals 685 and 687 as the plunger 681 is moved from the loaded position depicted in FIG. 8A (i.e., on the left end of the module 680) to the unloaded position (i.e., towards the exit port 682 as indicated by the arrow in FIG. 8A). The plunger 681 may preferably include an o-ring (depicted) or other sealing structure to prevent materials in the chambers from moving past the plunger 681 in the opposite direction, i.e., away from the opening 682.

FIG. 8B depicts a dispensing operation in which the plunger 681 is in transit from the loaded position of FIG. 8A to the unloaded position. In FIG. 8B, the tip 683 has pierced seal 685 such that the materials in chambers 684 and 686 can contact each other and mix. It may be preferred that chamber 684 contain a liquid 690, e.g., water, saline, etc. and that chamber 686 contain a dried-down reagent 692 (e.g., a lysing agent, fibrinogen, etc.), with the liquid 690 causing the reagent 692 to enter into a solution, suspension, mixture, etc. with the liquid 690. Although reagent 692 is depicted as being dried-down within chamber 686, it may be located in, e.g., a powder, gel, solution, suspension, or any other form. Regardless of the form of the materials in the chambers 684 and 686, piercing or opening of the seal 685 allows the two materials to contact each other and preferably mobilize within module 680 such that at least a portion can be delivered out of the module 680.

As the plunger 681 is advanced towards the exit port 682, the tip 683 also preferably pierces seal 687 such that the materials 694 in the chamber 688 can preferably contact the materials 690 and 692 from chambers 684 and 686.

When fully advanced towards the exit port 682, the tip 683 may preferably pierce exit seal 689 provided over exit port 682, thereby releasing the materials 690, 692 and 694 from fluid module 680 and into, e.g., a staging chamber or other space. It may be preferred that the shape of the plunger 681 and tip 683 mate with the shape of the final chamber 688 and exit port 682 such that substantially all of the materials in the various chambers are forced out of the fluid module 680 when the plunger 681 is advanced completely through the fluid module 680 (i.e., all of the way to the right of FIGS. 8A & 8B).

FIG. 8C is an enlarged view of on exemplary alternative tip 1683 in the opening 1682 of a module. The tip 1683 preferably extends from a plunger 1681. As discussed herein, the shape of the tip 1683 and plunger 1681 may preferably mate with the shape of the opening 1682 in the module housing 1695. For example, the portion of the depicted tip 1683 has a conical shape that conforms to the frusto-conical shape of the opening 1682. In addition, it may be preferred that the plunger 1681 and the inner surface 1696 of the module facing the plunger 1681 also conform to each other. Conformance between the plunger 1681 and tip 1683 with the mating features of the module may enhance complete delivery of materials from the module into the cartridges of the present invention.

Furthermore, it may be preferred that the tip 1683 be provided in a shape or with features that facilitate the transfer of materials past the seals pierced by the tip 1683. The feature may be as simple as a channel 1697 formed in an otherwise conical tip 1683 as depicted in FIGS. 8C & 8D. Alternatively, the tip 1683 itself may have many other shapes to reduce the likelihood that the tip will form a barrier to fluid flow with a seal it pierces. Such alternatives may include, e.g., star-shaped piercing tips, ridges, etc.

The plunger 681 in module 680 may be moved by any suitable actuator or technique. For example, the plunger 681 may be driven by a mechanical device (e.g., piston) inserted into module 680 through driver opening 698 or fluid pressure may be introduced into module 680 through driver opening 698 to move the plunger 681 in the desired direction. It may be preferred to drive the plunger 681 using, e.g., a stepper motor or other controlled mechanical structure to allow for enhanced control over the movement of plunger 681 (and any associated structure such as, e.g., tip 683). Other means for moving plunger 681 will be known to those skilled in the art, e.g., solenoid assemblies, hydraulic assemblies, pneumatic assemblies, etc.

The module 680, plunger 681 and tip 683 may be constructed of any suitable material or materials, e.g., polymers, metals, glasses, silicon, ceramics, etc. that provide the desired qualities or mechanical properties and that are compatible with the materials to be stored in the fluid modules. Similarly, the seals 685, 687 and 689 may be manufactured of any suitable material or materials, e.g., polymers, metals, glasses, etc. For example, the seals may preferably be manufactured from polymer film/metallic foil composites to provide desired barrier properties and compatibility with the various materials to be stored in the module 680.

It may be preferred that the materials used for both the seals and the module housing be compatible with the attachment technique or techniques used to attach the seals in a manner that prevents leakage between the different chambers. Examples of some attachment techniques that may be used in connection with modules 680 include, e.g., heat sealing, adhesives, chemical welding, heat welding, ultrasonic welding, combinations thereof, etc. It should also be understood that the modules may be constructed such that the seals are held in place by friction, compression, etc. Furthermore, it should be understood that in some embodiments, it may be possible to open the seals in a fluid module without the use of tip or other structure that pierces the seals. For example, the seals may be opened through fluid pressure alone (i.e., the seals may be designed to burst under pressure as the plunger is moved from the loaded position towards the exit port using, e.g., a line of weakness formed in the seal, etc.).

Sensor Considerations

The systems and methods of the present invention may preferably detect the presence of target biological analyte in a test sample through the use of acousto-mechanical energy that is measured or otherwise sensed to determine wave attenuation, phase changes, frequency changes, and/or resonant frequency changes.

The acousto-mechanical energy may be generated using, e.g., piezoelectric-based surface acoustic wave (SAW) devices. As described in, e.g., U.S. Pat. No. 5,814,525 (Renschler et al.), the class of piezoelectric-based acoustic mechanical devices can be further subdivided into surface acoustic wave (SAW), acoustic plate mode (APM), or quartz crystal microbalance (QCM) devices depending on their mode of detection.

In some embodiments, the systems and methods of the present invention may be used to detect a target biological analyte attached to a detection surface. In other embodiments, the devices may be used to detect a physical change in a liquid (e.g., an aqueous solution), such as, e.g., a change in viscosity, that is indicative of the presence of the target biological analyte. The propagation velocity of the surface wave is a sensitive probe that may be capable of detecting changes such as mass, elasticity, viscoelasticity, conductivity and dielectric constant in a medium in contact with the detection surface of an acousto-mechanical sensor. Thus, changes in one or more of these (or potentially other) physical properties may result in changes in the attenuation of the surface acoustic wave.

APM devices operate on a similar principle to SAW devices, except that the acoustic wave used can be operated with the device in contact with a liquid. Similarly, an alternating voltage applied to the two opposite electrodes on a QCM (typically AT-cut quartz) device induces a thickness shear wave mode whose resonance frequency changes in proportion to mass changes in a coating material.

The direction of the acoustic wave propagation (e.g., in a plane parallel to the waveguide or perpendicular to the plane of the waveguide) may be determined by the crystal-cut of the piezoelectric material from which the biosensor is constructed. SAW biosensors in which the majority of the acoustic wave propagates in and out of the plane (e.g., Rayleigh wave, most Lamb-waves) are typically not employed in liquid sensing applications because of acoustic damping from the liquid in contact with the surface.

For liquid sample mediums, a shear horizontal surface acoustic wave biosensor (SH-SAW) may preferably be used. SH-SAW sensors are typically constructed from a piezoelectric material with a crystal-cut and orientation that allows the wave propagation to be rotated to a shear horizontal mode, i.e., parallel to the plane defined by the waveguide, resulting in reduced acoustic damping loss to a liquid in contact with the detection surface. Shear horizontal acoustic waves may include, e.g., thickness shear modes (TSM), acoustic plate modes (APM), surface skimming bulk waves (SSBW), Love-waves, leaky acoustic waves (LSAW), and Bleustein-Gulyaev (BG) waves.

In particular, Love wave sensors may include a substrate supporting a SH wave mode such as SSBW of ST quartz or the leaky wave of 36° YXLiTaO₃. These modes may preferably be converted into a Love-wave mode by application of thin acoustic guiding layer or waveguide. These waves are frequency dependent and can be generated if the shear wave velocity of the waveguide layer is lower than that of the piezoelectric substrate.

Waveguide materials may preferably be materials that exhibit one or more of the following properties: low acoustic losses, low electrical conductivity, robustness and stability in water and aqueous solutions, relatively low acoustic velocities, hydrophobicity, higher molecular weights, highly cross-linked, etc. In one example, SiO₂ has been used as an acoustic waveguide layer on a quartz substrate. Examples of other thermoplastic and crosslinked polymeric waveguide materials include, e.g., epoxy, polymethylmethacrylate, phenolic resin (e.g., NOVALAC), polyimide, polystyrene, etc.

Other potentially suitable materials and constructions for use with acousto-mechanical sensors used in the detection cartridges of the present invention may be described in, e.g., PCT Application No. ______, titled “Acoustic Sensors and Methods”, filed on even date herewith (Attorney Docket No. 60209WO003).

Alternatively, QCM devices can also be used with liquid sample mediums. Biosensors employing acousto-mechanical devices and components may be described in, e.g., U.S. Pat. No. 5,076,094 (Frye et al.); U.S. Pat. No. 5,117,146 (Martin et al.); U.S. Pat. No. 5,235,235 (Martin et al.); U.S. Pat. No. 5,151,110 (Bein et al.); U.S. Pat. No. 5,763,283 (Cernosek et al.); U.S. Pat. No. 5,814,525 (Renschler et al.); U.S. Pat. No. 5,836,203 ((Martin et al.); and U.S. Pat. No. 6,232,139 (Casalnuovo et al.). Shear horizontal SAW devices can be obtained from various manufacturers such as Sandia Corporation, Albuquerque, New Mexico. Some SH-SAW biosensors that may be used in connection with the present invention may also described in Branch et al., “Low-level detection of a Bacillus anthracis simulant using Love-wave biosensors on 36° YX LiTaO₃,” Biosensors and Bioelectronics (accepted 22 Aug. 2003).

As used herein and in the appended claims, the singular forms “a,” “and,” and “the” include plural referents unless the context clearly dictates otherwise. Thus, for example, reference to “a” or “the” component may include one or more of the components and equivalents thereof known to those skilled in the art.

All references and publications cited herein are expressly incorporated herein by reference in their entirety into this disclosure. Exemplary embodiments of this invention are discussed and reference has been made to some possible variations within the scope of this invention. These and other variations and modifications in the invention will be apparent to those skilled in the art without departing from the scope of the invention, and it should be understood that this invention is not limited to the exemplary embodiments set forth herein. Accordingly, the invention is to be limited only by the claims provided below and equivalents thereof.

1. A detection cartridge comprising:
a housing comprising an interior volume;
a sensor operably attached to the housing, the sensor comprising a detection surface;
a detection chamber located within the interior volume of the housing, wherein the detection chamber comprises a volume defined by the detection surface and an opposing surface spaced apart from and facing the detection surface, wherein the opposing surface comprises a flow front control feature; and
a waste chamber located within the interior volume of the housing, the waste chamber in fluid communication with the detection chamber.

2. A cartridge according to claim 1, wherein the detection surface comprises an acousto-mechanical waveguide.

3. A cartridge according to claim 1, wherein the sensor comprises a surface acoustic wave acousto-mechanical sensor.

4. A cartridge according to claim 1, wherein the flow front control feature comprises discrete structures protruding from and separated by a land area on the opposing surface of the detection chamber.

5. A cartridge according to claim 1, wherein the flow front control feature comprises one or more channels in the opposing surface of detection chamber.

6. A cartridge according to claim 5, wherein at least one channel of the one or more channels is oriented generally perpendicular to a longitudinal axis defined within the detection chamber between an input end and an output end of the waste chamber.

7. A cartridge according to claim 1, wherein the flow front control feature comprises one or more regions of hydrophobic material occupying a portion of the opposing surface and one or more regions of hydrophilic material occupying a portion of the opposing surface.

8. A cartridge according to claim 7, further comprising at least one pair of successive bands of hydrophobic material and hydrophilic material wherein each pair of successive bands extends across a width of the detection chamber.

9. A cartridge according to claim 1, wherein the flow front control feature comprises discrete structures protruding from and separated by a land area on the opposing surface of the detection chamber, one or more regions of hydrophobic material occupying a portion of the opposing surface, and one or more regions of hydrophilic material occupying a portion of the opposing surface.

10. A cartridge according to claim 1, wherein the flow front control feature comprises one or more channels in the opposing surface of detection chamber, one or more regions of hydrophobic material occupying a portion of the opposing surface, and one or more regions of hydrophilic material occupying a portion of the opposing surface.

11. A cartridge according to claim 1, further comprising absorbent material located within the waste chamber.

12. A cartridge according to claim 1, wherein the cartridge further comprises capillary structure located between the detection chamber and the waste chamber.

13. A cartridge according to claim 1, further comprising a vent that, when open, places the interior volume of the housing in fluid communication with ambient atmosphere around the cartridge.

14. A cartridge according to claim 13, wherein the vent is located in the waste chamber.

15. A cartridge according to claim 13, wherein the vent comprises a closure element.

16. A cartridge according to claim 1, further comprising a fluid monitor operably connected to the housing, wherein liquid located within the interior volume of the housing can be sensed by the fluid monitor.

17. A detection cartridge comprising:
a housing comprising an interior volume;
a sensor operably attached to the housing, the sensor comprising surface acoustic wave acousto-mechanical sensor;
a detection chamber located within the interior volume of the housing, wherein the detection chamber comprises a volume defined by the detection surface and an opposing surface spaced apart from and facing the detection surface, wherein the opposing surface comprises one or more channels formed therein;
a waste chamber located within the interior volume of the housing, the waste chamber in fluid communication with the detection chamber;
absorbent material located within the waste chamber; and
capillary structure located between the detection chamber and the waste chamber.

18. A detection cartridge comprising:
a cartridge housing comprising an interior volume;
a sensor operably attached to the cartridge housing, the sensor comprising a detection surface;
a detection chamber located within the interior volume of the cartridge housing, wherein the detection chamber comprises a volume defined by the detection surface and an opposing surface spaced apart from and facing the detection surface, wherein the opposing surface comprises a flow front control feature;
a waste chamber located within the interior volume of the cartridge housing, the waste chamber in fluid communication with the detection chamber;
one or more sealed modules, wherein each module of the one or more sealed modules comprises an exit port attached to the cartridge housing through one or more module ports that open into the interior volume of the cartridge housing, and wherein each module further comprises:
a module housing comprising an exit port and a sealed interior volume;
an exit seal located over the exit port of the module; and
a plunger located within the interior volume of the module housing, wherein the plunger is movable from a loaded position in which the plunger is distal from the exit port to an unloaded position in which the plunger is proximate the exit port;
wherein movement of the plunger towards the exit port opens the exit seal such that material from the interior volume of the module housing exits through the exit port into the interior volume of the cartridge housing.

19. A cartridge according to claim 18, further comprising a staging chamber within the interior volume of the cartridge housing, wherein the staging chamber is located upstream from the detection chamber, and wherein the module ports open into the staging chamber.

20. A cartridge according to claim 18, wherein the interior volume of at least one module of the one or more sealed modules comprises:
a first chamber comprising a liquid located therein;
a second chamber located within the interior volume of the module housing, the second chamber comprising a reagent located therein; and
an inter-chamber seal isolating the second chamber from the first chamber within the module housing;
wherein the first chamber, the inter-chamber seal, and the second chamber are located between the plunger and the exit seal;
wherein movement of the plunger towards the exit port opens the inter-chamber seal such that the liquid in the first chamber contacts the reagent in the second chamber.

21. A method of moving sample material through the detection cartridge of claim 1, the method comprising:
providing a detection cartridge according to claim 1;
delivering sample material into the interior volume of the housing of the detection cartridge, wherein the sample material flows into the detection chamber, and wherein flow front progression of the sample material through the detection chamber and towards the waste chamber is controlled at least in part by the flow front control feature on the opposing surface within the detection chamber.

22. A method according to claim 21, wherein delivering sample material into the detection chamber comprises delivering the sample material into a staging chamber located within the interior volume of the housing, wherein the sample material flows from the staging chamber into the detection chamber.

23. A method according to claim 21, wherein detection cartridge further comprises absorbent material within the waste chamber, and wherein the absorbent material draws sample material into the waste chamber.

24. A method according to claim 21, further comprising capillary structure located between the detection chamber and the waste chamber, wherein the capillary structure draws sample material from the detection chamber.

25. A method according to claim 21, further comprising a vent that, when open, places the interior volume of the housing in fluid communication with ambient atmosphere around the cartridge, and wherein the method comprises opening the vent to control sample material flow through the detection chamber.

26. A method according to claim 25, wherein opening the vent comprises adjusting the size of the vent to adjust the rate of sample material flow through the detection chamber.

27. A sealed module comprising:
a housing comprising an exit port and a sealed interior volume;
an exit seal located over the exit port;
a first chamber located within the interior volume of the housing, the first chamber comprising a liquid located therein;
a second chamber located within the interior volume of the housing, the second chamber comprising a reagent located therein;
an inter-chamber seal isolating the second chamber from the first chamber within the housing; and
a plunger, wherein the first chamber, the inter-chamber seal, the second chamber, and the exit seal are located between the plunger and the exit port, and wherein the plunger is movable from a loaded position in which the plunger is distal from the exit port to an unloaded position in which the plunger is proximate the exit port;
wherein movement of the plunger towards the exit port opens the inter-chamber seal such that the liquid in the first chamber contacts the reagent in the second chamber, and wherein further movement of the plunger into the unloaded position opens the exit seal such that the liquid and the reagent from the interior volume of the housing exit through the exit port.

28. A module according to claim 27, wherein the plunger comprises a tip, wherein the tip faces the inter-chamber seal and wherein the tip pierces the inter-chamber seal to open the inter-chamber seal.

29. A module according to claim 27, wherein the first chamber and the second chamber comprise hermetically sealed compartments.

30. A module according to claim 27, wherein the plunger defines a portion of a volume of the first chamber when the plunger is in the loaded position.

31. A module according to claim 27, wherein the plunger mates with the exit port when the plunger is in the unloaded position.

32. A module according to claim 27, wherein the liquid in the first chamber comprises a water and the reagent in the second chamber comprises a hydrolyzable material.

33. A method of delivering materials using a sealed module of claim 27, the method comprising:
providing a sealed module according to claim 27;
moving the plunger towards the exit port of the sealed module to open the inter-chamber seal and force the liquid from the first chamber into contact with the reagent in the second chamber; and
moving the plunger towards the exit port to open the exit seal and expel the liquid and the reagent from the interior volume of the housing through the exit port.

34. A method according to claim 33, wherein the plunger comprises a tip that pierces the inter-chamber seal.

35. A module comprising:
a housing comprising an exit port and a sealed interior volume;
an exit seal located over the exit port;
a chamber located within the interior volume of the housing, the chamber comprising one or more reagents located therein;
a plunger movable from a loaded position in which the plunger is distal from the exit port to an unloaded position in which the plunger is proximate the exit port; and
an input port in fluid communication with the chamber, wherein the input port enters the chamber between the plunger and the exit port when the plunger is in the loaded position;
wherein movement of the plunger towards the exit port opens the exit seal such that material from the interior volume of the housing exits through the exit port.

36. A module according to claim 35, further comprising a seal closing the input port.

37. A module according to claim 35, wherein the plunger comprises a tip, wherein the tip faces the exit seal and wherein the tip pierces the exit seal to open the exit seal.

38. A module according to claim 35, wherein the plunger defines a portion of a volume of the chamber when the plunger is in the loaded position.

39. A module according to claim 35, wherein the plunger mates with the exit port when the plunger is in the unloaded position.

40. A method of delivering materials using a module according to claim 35, the method comprising:
providing a module according to claim 35;
delivering sample material comprising a liquid into the chamber through the input port, wherein the sample material contacts the reagent located within the chamber; and
moving the plunger towards the exit port to open the exit seal such that the liquid exits from the chamber through the exit port.
